(19) United States
(12) Patent Application Publication
(10) Pub. No.: US 2007/0048351 A1
(43) Pub. Date: Mar. 1, 2007

(54) Drugs coated on a device to treat vulnerable plaque

(75) Inventor: Anthony C. Lunn, Princeton, NJ (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 11/505,875

(22) Filed: Aug. 18, 2006

Related U.S. Application Data
(60) Provisional application No. 60/712,817, filed on Sep. 1, 2005.

(51) Int. Cl. A61F 2/06; A61K 31/727; A61K 31/56; A61K 31/401; A61K 31/366; A61K 31/22

(52) U.S. Cl. 424/423; 623/1.11; 514/56; 514/423; 514/182; 514/460; 514/548

(57) ABSTRACT

A treatment for vulnerable plaque may include a device having a polymeric coating that contains a statin and/or one or more other drugs, where the statin and/or one or more other drugs may be locally released in a sustained fashion at the point of insertion of the device. The coating may coat a self-expanding or balloon expanding structure, such as a fibrous or thin-film structure, intended to reduce the occurrence or severity of restenosis. The coating may be applied to a gently expanding device that reduces vessel trauma by virtue of exerting a low force of expansion against the vessel wall. The coating may be an absorbable polymer on an implantable device such that the absorbable polymer coating degrades at a specified rate to reduce the risk of “late” thrombosis. An anti-thrombogenic agent may also be disposed on or in the absorbable polymer coating to eliminate residual polymer on the surface after the coating is absorbed.

### FIELD OF THE INVENTION

The present invention pertains to the treatment of vulnerable plaque by delivery of a drug such as a statin by way of a device that can be inserted or implanted in a vessel.

### BACKGROUND OF THE INVENTION

Applicant makes no admission that any of the following cited articles and methods are prior art, and expressly reserves the right to demonstrate, where appropriate, that these articles and methods do not constitute prior art under the applicable statutory provisions.

Over the past decade, increasing attention has been paid to coronary diseases caused by inflammatory processes that lead to the rupture of “vulnerable plaques.” (See Monroe et al, J. Am. College Cardiol. 41: 23S-30S at 24S; Naghavi et al, Circulation 108: 1664-72 (2003).) Vulnerable plaque typically consists of a lipid-rich core covered by a thin layer of inflammatory cells and is not necessarily associated with vascular stenosis as found in arteries clogged with calcified plaque. Smooth muscle cell (SMC) apoptosis, loss of extracellular matrix (ECM integrity, and inflammatory cell accumulation in the fibrous cap are thought to be important pathogenic factors leading to plaque instability. (Kolodgie et al, Curr Opin Cardiol. 16: 285-92 (2001).) When the layer of inflammatory cells erodes or ruptures in response to mechanical stress or other factors, the lipid pool is exposed to the blood flow, causing clots to form in the artery. These clots may grow rapidly and block the artery or detach and travel downstream, leading to thromboembolic events, unstable angina, myocardial infarction, and/or sudden death. Recent studies suggest that such plaque rupture may trigger at least 60 to 70% of all fatal myocardial infarctions.

Traditional atherosclerosis therapies, like balloon angioplasty and stenting, are not appropriate for the treatment of vulnerable plaque. Stents have an additional disadvantage of inducing intimal hyperplasia, the uncontrolled migration and proliferation of medial smooth muscle cells through the openings of the expanded stent meshes, ultimately resulting in restenosis of the arterial wall.

Vulnerable plaque can be a type of plaque that may rupture, fracture or erode thereby causing a thrombosis. A common type of vulnerable plaque includes a thin fibrous cap and large lipid core. In other words, this is a soft plaque that is vulnerable to sudden rupture. In addition, this type of vulnerable plaque is hidden within the arterial wall, not visibly blocking the artery. The rupture of this type of vulnerable plaque can cause the release of the plaque's contents, a liquid pool of fat, cholesterol, and other debris into the blood stream, where they quickly coagulate to form a blood clot that can block blood flow to the heart and cause a heart attack.

Needs exist for new treatments for vulnerable plaque by local delivery of agents which reduce the likelihood of plaque rupture.

### SUMMARY OF THE INVENTION

Embodiments of the present invention solve the problems and/or overcome the drawbacks and disadvantages of the prior systems by providing one or more drugs at the site of a vulnerable plaque for treatment of the vulnerable plaque.

In particular, the present invention accomplishes this by providing a drug for treatment of vulnerable plaques within a coating on an implantable device for inserting into a body lumen of an individual. Embodiments of the present invention may include a method of treating vulnerable plaque including inserting an implantable device into a body lumen of an individual. The device may include a generally tubular body having a contracted state and an enlarged state. The generally tubular body may be an interconnecting structure with pores substantially along the length of the generally tubular body, expandable from the contracted state to the enlarged state, and sufficiently flexible such that the generally tubular body conforms to a contour of an inner surface of the body lumen of an individual. The device may include a therapeutically effective amount of a drug selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an antithrombotic drug, a drug that promotes healing and re-endothelialization, and combinations thereof.

The generally tubular body may be self-expandable. The generally tubular body may include a plurality of filaments coherently engaged by braiding, weaving, or knitting. Alternatively, the generally tubular body may be expanded by other means, such as by a balloon, etc.

Pore size on the generally tubular body is preferably no greater than about 500 microns.

The interconnecting structure of the generally tubular body may include a plurality of polymer or metallic microfilaments. The drug may be contained within a plurality of polymer microfilaments or within cavities created by a plurality of metallic microfilaments.

Alternatively, the drug may be contained in a polymer coating. The polymer coating preferably coats the interconnecting structure. In a preferred embodiment of the present invention the polymer coating may be coated with a second polymer coating that increases or decreases the release rate of the drug.

The implantable device may be inserted into a body lumen of an individual at a site of a vulnerable plaque, and the device may include a therapeutically effective amount of a statin.

The implantable device may be coated with an absorbable polymer coating, and the absorbable polymer coating may degrade within the individual at a pre-determined rate. The implantable device may contain a top coating including absorbable polymer material and an anti-thrombogenic drug.

Additional features, advantages, and embodiments of the invention may be set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### DETAILED DESCRIPTION OF PREFERRED EMBODIMENTS

Embodiments of the present invention may include a treatment for vulnerable plaque with a device having a polymeric coating that contains a statin and/or one or more other drugs, where the statin and/or one or more other drugs may be locally released in a sustained fashion at the point of insertion of the device. The coating may coat a self-expanding or balloon expanding structure, such as a fibrous or thin-film structure, intended to reduce the occurrence or severity of restenosis. The coating may be applied to a gently expanding device that reduces vessel trauma by virtue of exerting a low force of expansion against the vessel wall. A drug coating may be disposed under an absorbable polymer coating on an implantable device such that the absorbable polymer coating degrades at a specified rate to reduce the risk of “late” thrombosis”.

Statins are a class of drugs that competitively inhibit 3-hydroxy-3-methylglutaryl-coenzyme A (HMG-CoA) reductase, the enzyme that catalyzes the rate-limiting step in cholesterol biosynthesis. HMG-CoA reductase inhibition results in a systematic reduction of the formation of cholesterol in the liver and blood. (Maron et al., Circulation 101: 207-13 (2000) at 207.) The class of statin drugs may be derived from fungal fermentation (e.g., lovastatin, simvastatin and pravastatin) or made synthetically (e.g., fluvastatin, atorvastatin and cerivastatin). Statins vary in their physical properties. For example, lovastatin, simvastatin, atorvastatin and cerivastatin are hydrophobic, whereas pravastatin is hydrophilic, and fluvastatin has an intermediate hydrophobicity. (Id.) Various salts and analogues of statins are well known in the art.

Statins affect a number of physiological responses in addition to reducing cholesterol. Statins slow the progression and induce the regression of coronary atherosclerosis, reduce the formation of new lesions, and reduce the incidence of coronary events (Maron at 209.) Although the magnitude of the regression of coronary atherosclerosis in response to statins is relative minor, statins provide a marked clinical benefit in reducing cardiovascular events and death. This observation suggests that statins stabilize, as well as reduce, both stable and vulnerable arterial plaques. (See Maron at 209). Statins also inhibit proliferation and migration of smooth muscle cells (SMCs) (Negre-Aminou et al, Biochim. Biophys. Acta 1345: 259-68 (1997), enhance endothelial function (see Maron at 209), including the promotion of collagen accumulation (Rabbani et al, Cariovascular Res. 41: 402-17 (1999) at 403), and decrease macrophage proliferation, including those active in vulnerable plaque lesions (Id. at 405). Other drugs, including beta-adrenergic blocking agents and possibly angiotensin-converting enzyme inhibitors and antioxidants, may also reduce the incidence of plaque rupture. (Tanabe et al., Curr. Pharmaceutical Design 10: 357-67 (2003)). Antioxidants may promote plaque stabilization by reducing extracellular matrix (ECM) degradation, as may promoters of extracellular matrix (ECM) synthesis or other inhibitors of ECM degradation (Rabbani et al, Cardiovascular Res. 41: 402-17 (1999) at 405-408). These additional drugs may also be used in accord with the principles of this invention.

A “statin” is preferably defined herein to be an inhibitor of HMG-CoA reductase that contains a moiety that can exist either as a 3-hydroxy lactone ring (in an inactive form) or as the corresponding ring opened dihydroxy open acid, as shown in formulae I and II, respectively (see U.S. Pat. No. 6,777,552):

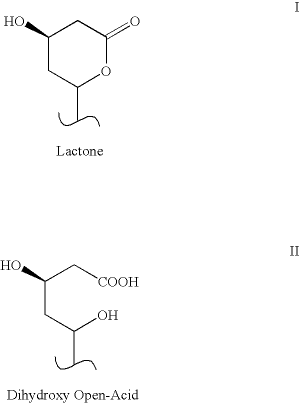

Statins in accordance with the principles of the invention include, but are not limited to, lovastatin (U.S. Pat. No. 4,231,938), simvastatin (ZOCOR; U.S. Pat. No. 4,444,784 and WO 00/53566), atorvastatin (LIPOTOR; U.S. Pat. No. 5,969,156), cerivastatin (U.S. Pat. No. 5,006,530 and U.S. Pat. No. 5,177,080) (which is less preferred and presently has been withdrawn from market), rosuvastatin (U.S. Pat. No. RE37,314), pitavastatin, BMY 22089 (G.B. Patent No. 2,202,846); pravastatin (PRAVACHOL; U.S. Pat. No. 4,346,227), and fluvastatin (LESCOL; U.S. Pat. No. 4,739,073). Structures of the aforementioned representative statins are shown in Schachter, Fund'l Clin. Pharmacol. 19: 117-25 at FIG. 2, which is herein incorporated by reference.

Other statins suitable for the present invention include, but are not limited to, mevastatin (U.S. Pat. No. 3,983,140), velostatin (U.S. Pat. No. 4,448,784 and U.S. Pat. No. 4,450,171), compactin (U.S. Pat. No. 4,804,770), dalvastatin (U.S. Pat. No. 5,733,558), bervastatin (U.S. Pat. No. 5,082,859), dihydrocompactin (U.S. Pat. No. 4,450,171), ZD-4522 (U.S. Pat. No. 5,260,440), and NK-104 (U.S. Pat. No. 5,102,888). Statins may also include pharmacologically active salts, such as sodium salts, calcium salts (U.S. Pat. No. 6,777,552), dihydroxy open acid salt forms (U.S. Pat. No. 6,569,461) and other derivatives, such as ester derivatives (see e.g., U.S. Pat. No. 6,294,680; U.S. Pat. No. 6,777,552). HMG-CoA reductase inhibitors can be identified readily using well-known assays. For example, see the assays described or cited in U.S. Pat. No. 4,231,938 at col. 6 and WO 84/02131 at 30-33. Formulations and treatment modalities according to the present invention may be tested for safety and efficacy using animal models, including murine and porcine models. (See Majesky, Circulation 105: 2010 (2002) and U.S. Pat. No. 6,580,016, respectively.)

The choice of statin or statins to be used according to the present invention is preferably guided by an activity displayed by a statin that improves overall clinical outcome. In one aspect of the present invention, statins are selected for their ability to passivate plaque, particularly vulnerable plaque, where plaque passivation is preferably defined as remodeling vulnerable plaque composition to reduce the risk of plaque rupture or thrombosis. In one particular embodiment, a statin may be delivered in an amount effective to reduce lipids in the core of vulnerable plaque and/or to increase the thickness of its fibrous cap. While the applicability of the present invention is not limited by theory, it is believed that some of this effect of statins is mediated by inhibition of hepatocyte HMG Co-A reductase. In this embodiment, the device may serve as a depot for delivery of statin. In one aspect of this embodiment, the device may double as a mechanical shield over the eroded coronary surface and optionally as a stent to improve luminal area.

The present invention may include a device with statins, beta-adrenergic blocking agents, angiotensin converting enzyme (ACE) inhibitors and antioxidants or other drugs for local release or release into the blood stream. The device may be placed at or near a vascular lesion or proximally upstream of a vascular lesion, where the vascular legion may be vulnerable plaque. In one aspect of the invention, a statin and/or one or more other drugs in accordance with the principles of the invention may be administered to a patient by the device in a therapeutically effective amount to passivate vulnerable plaque, i.e., to change beneficially the composition of the lesion. In another aspect of the invention, the device may provide a statin and/or one or more other drugs in accordance with the principles of the invention at a therapeutically effective amount to reduce the occurrence of plaque rupture or otherwise improves clinical outcome in a patient.

The device may be used in a method of treating vulnerable plaque including implantation, placement or insertion of the device at the site of vulnerable plaque to be treated. In this embodiment, the device may be capable of delivering a statin and/or one or more other drugs locally to surrounding tissue. In this aspect of the invention, the device may serve an additional beneficial effect of covering or shielding the vulnerable plaque to prevent or reduce the incidence or harmful effects of plaque rupture and/or detachment. When the device is used to deliver a statin locally to tissues at or surrounding vulnerable plaque, preferred statins are hydrophobic or moderately hydrophobic, as they are expected to have a longer residence time in the surrounding tissue. When the device is used as a stent, it may include a drug that reduces SMC proliferation to reduce neointimal hyperplasia associated with the implantation of the stent. In another embodiment, the device may include a drug that promotes SMC proliferation to thicken the fibrous cap of vulnerable plaque, thereby reducing the risk that the plaque will rupture. In an alternative embodiment, wherein the drug is delivered into circulation, the device may be inserted or placed in a blood vessel, but is not necessarily inserted at the site of vulnerable plaque. The device also may be used in a method of treating vulnerable plaque as a depot for delivery of a statin and/or one or more other drugs into the arterial wall especially into the vulnerable plaque, and optionally into circulation, over a period of time.

The device can have one or more drugs. For example, the device of the present invention can contain drugs within it or be coated with a polymer. Such a polymer coating may be made either by coating the wires or struts of the device with methods known in the art, including spray or dip coating, or by treating an electrospun covering with heat or chemicals. The fibers created by an electrospinning process may have diameters averaging less than about 100 micrometers. The polymer may be mixed or combined with drugs immobilized within the polymer, including, alone or in combination, a statin, ACE inhibitor, statin-containing microspheres or ACE inhibitor microspheres. Optionally, the polymer of the device or coating may be biodegradable. Alternatively, a polymer need not be used.

In one embodiment, a tubular device preferably self-expands, resulting in a gentle pressure against the arterial wall that reduces the occurrence or severity of intimal hyperplasia by minimizing intimal trauma. A device that “gently expands” exerts a pressure against the internal wall of the bodily cavity only as high as required to prevent the device from becoming dislodged. Gentle expansion may be accomplished in other ways, such as by balloon expansion. The radial expansive force of the self-expanding device can be created by a plurality of filaments coherently engaged together to form a tube shape, for example, by braiding, weaving, or knitting. Alternatively, the device can be a self-expanding metallic or polymeric tube. In both of these embodiments, the self-expandable device preferably includes a generally tubular body having a contracted state and an enlarged state. The generally tubular body may have a pore size that is, in one embodiment, no greater than about 500 microns, substantially along the length of the generally tubular body. The generally tubular body is preferably sufficiently flexible to conform to a contour of an inner surface of a body lumen.

The present invention provides a device that may include a statin and/or one or more other drugs as discussed herein and pharmacologically acceptable excipients, carriers, or diluents. In one embodiment, the device may include wires or struts or similar support elements, metal or otherwise, that are coated with a polymeric composition containing the statin and/or one or more other drugs in accordance with the principles of the invention. In another embodiment, the wires or struts or similar support elements may contain cavities or holes in which the statin and/or one or more other drugs in accordance with the principles of the invention are disposed. In another embodiment, the device may be in the form of a balloon that can be expanded against the walls of a vessel, where a polymeric composition including a statin and/or one or more other drugs in accordance with the principles of the invention coats a surface of the balloon. The device is preferably capable of insertion into a coronary artery or insertion into a similarly tubular body part of an animal or human being. The statin and/or one or more other drugs in accordance with the principles of the invention may be released from the polymeric coating over a period of time once the device is inserted. The statin and/or one or more other drugs in accordance with the principles of the invention coated on a device may be used in a method of passivation of plaque, particularly vulnerable plaque, where the structure or content of the plaque is changed to reduce the risk of rupture. In another aspect of the invention, the statin and/or one or more other drugs in accordance with the principles of the invention may reduce the occurrence of plaque rupture and improve clinical outcome. The device of the present invention additionally may be used as a stent to increase and/or maintain an increased arterial diameter, when it is desirable to reduce stenosis at the site of insertion. In another embodiment, the drug(s) in accordance with the principles of the invention may be applied uniformly or non-uniformly to the device to enhance efficacy of the treatment.

In another embodiment, the statin delivered by the device may passivate plaque by a direct effect on the tissue at or surrounding the vulnerable plaque. The ability of the device to supply a statin locally in this embodiment can provide an advantage to other conventional routes of statin administration (e.g., enteric deliver to the extent that local delivery allows a higher and more even concentration of statin to be delivered to its site of action, reducing the risk of harmful side-effects caused by the episodic systemic delivery. In this embodiment, it is preferably unnecessary for the statin to target hepatocyte HMG Co-A reductase. When local delivery of a statin is desired, a hydrophobic statin, such as lovastatin, simvastatin, atorvastatin or fluvastatin, is preferred because the statin is expected to have a longer residence time in the tissue immediately surrounding the implanted device. Conversely, hydrophilic statins, such as pravastatin, are less preferred in this embodiment.

In one such embodiment, the statin can be selected for its ability to promote endothelial cell function. Simvastatin and lovastatin are preferred statins for this embodiment. In yet another embodiment, the statin may be selected for the ability to reduce inflammatory responses at the site of the plaque. Atorvastatin and fluvastatin are two preferred statins for this embodiment. In accord with the present invention, statins may be delivered in an effective amount to reduce SMC proliferation and migration. Lovastatin, simvastatin, atorvastatin or fluvastatin are preferred for this application. Reducing local SMC proliferation or migration can be particularly useful when the recipient of the device is at risk of neointimal hyperplasia and the resulting restenosis caused by insertion of the device. When the risk of neointimal hyperplasia is low, the device may include a drug that increases SMC proliferation. The device may include more than one statin, especially when statins with different properties may confer synergistic benefits.

In one embodiment according to the present invention, another drug can be incorporated into the device alone or in combination with a statin and/or another drug in accordance with the principles of the invention. Such drug may be chosen particularly to enhance the benefit of the first statin or to produce an additional benefit, such as reducing hyperplasia or passivating the plaque by a mechanism independent of the first statin. In yet another embodiment, the drug can replace the statin all together, and the device does not include a statin. Additional drugs may be added to the device including a statin and/or one or more other drugs in accordance with the principles of the invention, as desired, to achieve additional beneficial effects.

Useful drugs that may reduce the risk of plaque rupture by exerting the same or independent activities as statins may include other lipid lowering drugs, antioxidants, β-adrenergic blockers and angiotensin converting enzyme (ACE) inhibitors. Drugs that promote ECM synthesis of stability, such as metalloproteinase inhibitors (e.g. MMP9 inhibitors), are may also be useful according to the present invention. In one embodiment, the drug may increase SMC proliferation to thicken the fibrous cap at the site of vulnerable plaque, thereby reducing the risk of plaque rupture. The drug may include gene therapy agents. Such agents may include antisense molecules and molecules that form double-stranded RNAs capable of selectively reducing the expression of particular genes through the intercellular generation of small interfering RNAs (“siRNAs”).

Other drugs may achieve these or other additional benefits. For example, such other drugs in accordance with the principles of the invention may be an anti-inflammatory immunomodulator, such as dexamethasone, prednisolone, interferon γ-1b, leflunomide, mycophenolic acid, mizoribine, cyclosporine or ABT-578. Alternatively, or in addition, another drug may be an anti-proliferative agent, such as sirolimus, tacrolimus, everolimus, QP-2, paclitaxel, actinomycin, methotrexate, angiopeptin, vincristine, mitomycine, an antisense molecule targeting an mRNA involved in proliferation (e.g. cmyc mRNA), ribozymes (e.g., RESTENASE), 2-chloro-deoxyadenosine, or PCNA ribozyme. Various useful drugs may include those that inhibit migration or modify the extracellular matrix, such as batimastat, prolyl hydroxylase inhibitors, halofuginone, C-proteinase inhibitors, or probucol. Useful drugs also may include those that promote healing and re-endothelialization at the site of the plaque, such as BCP671, VEGF, estradiols, nitrous oxide donors and EPC antibodies. 17-β-estradiol is particularly preferred because it is believed to lead to favorable vascular healing after injury, which could lead to stabilization of vulnerable plaque. (New et al, “Estrogen-eluting, phosphorylcholine-coated stent implantation is associated with reduced neointimal formation but no delay in vascular repair in a porcine coronary model,” Catheterization and Cardiovascular Interventions 57: 266-261 (2002).) Heparin may be another useful drug. These drugs may be used alone or in combination with a statin or as part of a composition including a plurality of drugs in accordance with the principles of the invention.

As used herein, the phrase “administering to a patient” means inserting a device according to the present invention to an individual. The administering may include the use of other devices that assist the insertion and implantation of a device, such as a catheter. The individual may be a human who is diagnosed as having vulnerable plaque, or the individual may be an animal (i.e., if the device is used in a veterinary application). Vulnerable plaque may be diagnosed in an individual by any means, including but not limited to one of the methods described at paragraph 5 of U.S. Published Application 2004/014322, which paragraph is incorporated herein by reference.

As used herein, the phrase “therapeutically effective amount” of any drug means the amount of a drug, which alone or in combination with other drugs, provides a benefit in the treatment or passivation of vulnerable plaque, when administered by a device according to the present invention. A therapeutically effective amount may be the amount of drug required for any beneficial effect related to plaque passivation or decreased risk of rupture, such as the amount of the drug needed to lower lipid levels or to reduce SMC proliferation. Newly available animal models for treatment of vulnerable plaque, including murine, rabbit and porcine models, may be used to determine a therapeutically effective amount of a drug according to the present invention. (See, e.g., Majesky, Circulation 105: 2010 (2002) and U.S. Pat. No. 6,580,016.) In all cases, a statin and/or other drug may be combined with a pharmaceutically acceptable excipient(s). Suitable pharmaceutically acceptable excipients are described below.

When the device is used as a depot for systemic delivery of a drug, the present invention advantageously may allow equivalent, or higher, doses of a drug to be delivered over a sustained period compared to the episodic delivery achieved by ingesting tablets, for example. The ability to deliver sustained systemic levels of a statin may reduce side effects and increase efficacy. The same or greater advantages can be achieved when the statin is locally delivered. In the latter case, a therapeutically effective amount of a statin may be lower than typically required when delivering statins systemically, reducing the risk of toxicity from breakdown products or other side effects. The same guidelines apply to choosing therapeutically effective amounts of another drug.

Another embodiment of the invention can include an antithrombotic drug. The invention can include a surface treatment or coating to inhibit thrombus formation, including, heparin. The surface treatment can contain an agent to enhance attachment such as plasminogen or albumin. A heparin-containing thromboresistant layer can be provided on a device to treat vulnerable plaque as described herein. The antithrombotic drug can be used alone or in combination with other aspects of the invention as described herein, and may be chemically bound to the surface or, optionally, be able to diffuse from the surface.

Current heparin coatings used on implanted stents follow a principal of “permanent” heparin surfaces. The permanent heparin surfaces on existing implanted stents are generally non-leaching and formed on a non-absorbable polymer substrate. The non-absorbable polymer substrate itself is formed on a metal stent.

However, permanent heparin surfaces may create further health problems for patients implanted with stents including the permanent heparin surfaces. For example, it is possible that the heparin may be degraded over time in whole or in part by enzymatic or other processes. The “permanent” heparin surface may degrade off the surface of the polymer, leaving the underlying polymer coating exposed on the medical device within the individual in perpetuity, potentially delaying endothelial coverage of the device.

There is some indication that exposed polymer coatings remaining on a metal stent may be causative agents of “late” (post 30 days) stent thrombosis. If this analysis is correct, there may be long term potential health benefit to exposed bare metal surfaces on stents in place of exposed polymer coatings.

An embodiment of the present invention may include an improved heparin surface on a permanently implanted medical device made of metallic or other material.

The improved heparin surface may be created by coating an implantable device with an absorbable polymer, which may have a heparin surface on the surface of and/or in the absorbable polymer. Preferably, the heparin does not leach from the absorbable polymer while the polymer is undegraded. Therefore, the heparin preferably remains active on the surface of the absorbable polymer to inhibit formation of thrombus, resulting in an improvement over existing heparin coatings. In a preferred embodiment, the heparin molecules may be covalently bound to the polymer or to the metal via a coupling moeity.

In a preferred embodiment of the present invention, the polymer preferably degrades by hydrolysis. The polymer remain in place for a time period beyond that required for coverage of the stent struts by tissue, which initially will be proteinaceous and may, most desirably, become covered with functional endothelial cells. The time period for coverage of stent struts by tissue can be in excess of one month in humans. The polymer preferably degrades before “late” thrombosis has the potential to occur. Preferably, the degradation of the polymer occurs before the discontinuation of systemic antiplatelet or anticoagulant therapy, which is typically discontinued after several months.

After degradation and disappearance of the polymer, the implanted device no longer has a polymer coating. If the device is a metallic stent, the device remains within the individual with a pure metal exposed surface, reducing complications resulting from exposed non-absorbable polymer coatings.

The advantages of the present invention are best realized when the device is placed directly over vulnerable plaque, and preferably where the device containing the drugs also acts as a mechanical shield over vulnerable plaque to reduce the risk of rupture or to contain clots or emboli formed following rupture. Preferably, the device exerts a sufficient force against the lumen to keep the device in place against the pressure of flowing blood. However, it is preferable that the force against the lumen is sufficiently low to decrease or prevent restenosis resulting from excessive hyperplasia caused by implantation injury.

In one embodiment, the device may be inserted using gentle pressure to press the device gently against the vessel wall in an effort to reduce trauma. For example, the device may be balloon expandable. For the purpose of the present invention, a device that “gently expands” has an initial internal diameter that increases once placed in an appropriate bodily cavity, such that the pressure exerted by the device against the internal wall of the bodily cavity is only as high as required to prevent the device from becoming dislodged. A procedure to manufacture suitable self-expanding devices according to one embodiment of the present invention is disclosed in U.S. Published Application 2005/0038503, the disclosure of which is incorporated herein by reference in its entirety. The radial force of the self-expanding device is due, in part, to a plurality of filaments coherently engaged together to form a tube shape, for example, by braiding, weaving, or knitting, as described in U.S. Published Application 2005/0038503. The filaments may be composed of an elastic polymer, metal, or metal-polymer composite, including nitinol, stainless steel, platinum, or elgiloy, and may typically be about 12-25 microns in thickness. Such filaments may be biostable or biodegradable or biosorbable.

A variety of different combinations of filament diameters, filament components, and engaging styles may be used to achieve the self-expanding properties of the device. Although certain embodiments are described with reference to self-expanding embodiments, the principles described herein can be applied to non-self expanding or balloon expandable devices. Typically, the self-expanding device is annealed on a stainless steel mandrel fixture as described in U.S. Published Application 2005/0038503. The annealing at least partially determines the expanded diameter of the self-expanding device. For example, nitinol may be processed at about 500° C. for about 10-15 minutes with a mandrel of a desired diameter. In another example, stainless steel, elgiloy, or MP35n materials may be processed at temperatures of about 1000° C. for relatively longer periods, such as 2-4 hours. The resulting annealed device may then exhibit a desired expansion force to a desired diameter (again as primarily determined by the mandrel size). In another example, a sputtered nitinol film tube about 10-15 microns thick with stent laser hole micron pattern system may be used, ultimately creating a tube with a pore size of, for example, about 20-50 microns. In yet another example, a sputtered nitinol film tube about 10-15 microns thick with textured mandrel may be used, creating a folding film Generally with a prosthesis formed from a sputtered film, the sputtered film is sputtered directly onto a mandrel with a textured surface. The textured surface of the mandrel could be, for example, a cross-hatched pattern or a “waffle” type pattern that will allow the device to flex and expand more readily.

The self-expandable device includes a generally tubular body having a contracted state and an enlarged state. The generally tubular body may be sufficiently flexible to conform to a contour of an inner surface of said body lumen. At least one end of the device may be expandable to a greater diameter than a central region of the generally tubular body. Alternatively, at least one end may have a flared shape in the enlarged state. The generally tubular body may have a cone shape when it is in the contracted state.

In one embodiment, the generally tubular body may include of a plurality of microfilaments that interconnect to create a pore size no greater than about 500 microns substantially along the length of the generally tubular body. The microfilaments of the self-expandable device may be made by weaving, braiding or knitting. In another embodiment, the device may include a single microfilament that is woven, braided, or knitted to create a mesh with pore sizes no greater than about 500 microns substantially along the length of the generally tubular body. With the use of a single wire, the wire can be coated or impregnated with one or more drugs in accordance with the principles of the invention.

Alternatively, a thin film can be used having a micro-porous structure that can be gently expanded to gently press against the vessel wall without causing trauma. The thin film device can be formed using etching, extrusion, electro polishing, flat rolling and/or sputtering techniques, for example.

In another embodiment, the self-expandable device may have micropleats extending longitudinally along an axis of the generally tubular body, which may extend circumferentially along an axis of the generally tubular body. Alternatively, the generally tubular body in the contracted state may have a ribbon configuration, where gaps exist between the curls of the ribbon, and the generally tubular body in the expanded state has a ribbon configuration wherein no gaps exist between the curls of the ribbon.

The self-expandable device of this aspect of the invention may be used in a patient in need of a stent, as in the case where the patient suffers stenosis. In this case, the stent may be disposed internally to the generally tubular body of the self-expandable device, or the stent may be mounted on an external circumference of the generally tubular body. The stent may be integral with the generally tubular body or have a length less than the length of the generally tubular body. The microfilament portion of the general tubular body may be connected to the stent portion through at least one of welding, interweaving, interbraiding or integral forming, by a process described in more detail in U.S. Published Application 2005/0038503.

In another related embodiment, the device may include a polymeric composition in the form of a coating. The device in this embodiment may serve not only to give structural rigidity to the polymer coating, but to increase luminal flow by exerting a force against the vessel wall. In one embodiment, a coated stent may be used in a method to treat plaque that is also associated with stenosis, including vulnerable plaque that is associated with stenosis. In one embodiment, the coating may include a surface agent which reduces thrombosis. Heparin coatings or chemically modified heparin coatings have been successfully used in stents for this purpose. Lunn, “Heparin Stent Coatings,” in Endoluminal Stenting, U. Sigwart, ed., WB Saunders, London, (1996), herein incorporated by reference in its entirety). Other coatings that attract binding of plasminogen (e.g. epsilon-lysine), albumin, and/or extracellular matrix proteins (e.g. fibronectin, RGD peptide, or collagen) can enhance endothelial coverage.

A “coated device,” as used herein, is one wherein at least some of the individual members or wires that make up the device have a layer of polymer bonded to them; however, gaps between the structural support elements of the device or pores generally preferably remain open. This configuration offers advantages over “covered stents,” which use a polymer sleeve or sheath that encompasses or covers a portion of the stent and serves as a local drug delivery device. Particularly, coated devices according to the present invention may allow diffusion of drugs to both the blood stream and the tissue surrounding the device, which is particularly useful to improve the uniformity of drug delivery to local tissue, or when the device is used both as a depot and for local drug delivery. Further, the coating may help protect underlining endothelial cells against injury from the support elements of the device, the open pores or gaps may facilitate growth of endothelial cells over the device, and the coating may provide an appropriate surface for endothelial cells ultimately to cover the device. The polymer coating can be applied to the wire or strut surfaces of the device by means known in the art, including but not limited to dip coating, spray coating, or electrostatic spinning.

In one embodiment where the drugs can withstand certain temperatures, a manufacturing process disclosed in U.S. Published Application 2004/0051201, the disclosure of which is incorporated herein by reference in its entirety, can be used. The structural elements of the device may be covered with a fibrous, preferably electrospun, polymer that loses its ability to span the gaps between the structural elements of the device when the polymer is heated. The fibers are treated, e.g. by heating the covered device to a predetermined temperature, until at least some of the interstices are reduced. In one embodiment, the layer of fibers can coat a stent. The coated stent can be heated to a predetermined temperature for a predetermined time until the fibers bridging the supports of the stent collapse and bond to the stent. The fibers spanning the gaps may break and retract to the nearest wire by virtue of surface tension, so that the individual wires of the stent are coated. The coating can differ from that of a dip coating stent because the coating maintains a fibrous quality depending on the degree to which the stent was heated. The coating does not have to coat the entire circumference of the wire. Thus, the fibrous coating can be resistant to cracking and does not cause the individual wires to adhere to each other. In one embodiment, a drug that is temperature sensitive can be added to the device after the heating is completed. In this embodiment, the temperature sensitive drug may be spray-coated onto the fibrous coating, or the coated device may be dunked into a low viscosity solution containing the drug.

Accordingly, the device of the present invention can include a coating in which a statin and/or one or more other drugs in accordance with the principles of the invention are contained. The coating may contain a plurality of fibrils of a first polymer, the fibrils having an average diameter less than 100 microns that are adhered to an outside surface of the device and that are intertangled with each other but not woven. The statin and/or one or more other drugs in accordance with the principles of the invention may be dissolved within the fibrils, or contained in liquid or microsphere form within interstices defined by and located between the fibrils.

Many polymers are suitable for coatings of the device of the present invention, including polytetrafluoroethylene, polyglycolic acid/polylactic acid, polycaprolactone, polyhydroxybutyrate valerate, polyorthoester, polyethyleneoxide/polybutylene terephthalate, polyurethane, silicone, polyethylene terephthalate, polyvinyl pyrrolidone/cellulose esters, polyvinyl pyrrolidone/polyurethane, polymethylidene maloleate, polylactide/glycolide copolymers, polyethylene vinyl alcohol, polydimethyl siloxane (silicone rubber), and phosphorylcholine.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl-cellulose, alginates, gelatin, polyvinyl pyrrolidone, sucrose and acacia; humectants, such as glycerol; disintegrating agents, such as agar, calcium carbonate, potato and tapioca starch, alginic acid, certain silicates, EXPLOTAB, crospovidone, and sodium carbonate; solution retarding agents, such as paraffin; absorption accelerators, such as quaternary ammonium compounds: wetting agents, such as cetyl alcohol and glycerol monostearate; absorbents, such as kaolin and bentonite clay, lubricants, such as talc, calcium stearate, magnesium stearate, soil polyethylene glycols, and sodium lauryl sulfate; stabilizers, such as fumaric acid; coloring agents: buffering agents; dispersing agents; preservatives; organic acids; and organic bases. Additionally, many excipients may have more than one role or function, or be classified in more than one group; the classifications are descriptive only, and not intended to limit any use of a particular excipient.

The amounts and types of polymers and the ratio of various polymers in the inventive formulations are preferably selected to achieve a desired release profile of a statin and/or one or more other drugs in accordance with the principles of the invention. The polymer in which the drug is incorporated can be used to increase or decrease the release rate of the drug, and/or a polymer coating without drug can be applied on top of the polymer layer that contains the drug, in accord with principles known in the art. In one embodiment, slow drug release may be obtained by impregnating the polymer with microspheres containing a statin and/or one or more other drugs in accordance with the principles of the invention.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A method of treating vulnerable plaque comprising:
inserting an implantable device into a body lumen of an individual,
wherein the device comprises a generally tubular body having a contracted state and an enlarged state,
wherein the generally tubular body is an interconnecting structure with pores substantially along the length of the generally tubular body, expandable from the contracted state to the enlarged state, and sufficiently flexible such that the generally tubular body conforms to a contour of an inner surface of the body lumen of an individual, and
wherein the device comprises a therapeutically effective amount of a drug selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an anti-thrombogenic agent, a drug that promotes healing and re-endothelialization, and combinations thereof.

2. The method of claim 1, wherein the generally tubular body is self-expandable.

3. The method of claim 2, wherein the generally tubular body comprises a plurality of filaments coherently engaged by braiding, weaving, or knitting.

4. The method of claim 1, wherein pore size is no greater than about 500 microns.

5. The method of claim 1, wherein the interconnecting structure comprises a plurality of polymer or metallic microfilaments.

6. The method of claim 5, wherein the drug is contained within a plurality of polymer microfilaments.

7. The method of claim 5, wherein the drug is contained within cavities created by a plurality of metallic microfilaments.

8. The method of claim 1, wherein the drug is contained in a polymer coating.

9. The method of claim 8, wherein the polymer coating reduces thrombosis.

10. The method of claim 8, wherein the polymer coating coats the interconnecting structure.

11. The method of claim 8, wherein the polymer coating is coated with a second polymer coating that increases or decreases the release rate of the drug.

12. The method of claim 8, wherein the polymer coating is absorbable and degrades at a pre-determined rate.

13. The method of claim 1, wherein the device further comprises a top coating comprising an absorbable polymer and an anti-thrombogenic agent.

14. The method of claim 1, wherein the inserting an implantable device into a body lumen of an individual is at a site of a vulnerable plaque, and wherein the device further comprises a therapeutically effective amount of a statin.

15. A method of treating vulnerable plaque comprising:
inserting a device into an individual,
wherein the device comprises:
i) a body lumen support structure,
ii) a first polymer coating disposed on said body lumen support structure, and
iii) a therapeutically effective amount of a drug disposed within the first polymer coating, where the drug is selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an anti-thrombogenic agent, a drug that promotes healing and re-endothelialization, and combinations thereof.

16. The method of claim 15, wherein the body lumen support structure further comprises a plurality of fibrils having an average diameter less than about 100 microns, wherein the plurality of fibers are arranged in a substantially random pattern on the body lumen support structure so as to create a plurality of substantially random interstitial spaces within the body lumen support structure.

17. The method of claim 16, wherein the plurality of fibers are arranged in a substantially random pattern by a process comprising electrospinning.

18. The method of claim 15, wherein the first polymer coating is coated with a second polymer coating for increasing or decreasing a release rate of the drug from the first polymer coating.

19. The method of claim 15, wherein the device is a stent.

20. The method of claim 15, wherein the inserting a device into an individual is at a site of a vulnerable plaque, and wherein the device comprises a therapeutically effective amount of a statin.

21. The method of claim 20, wherein the statin promotes endothelial cell function.

22. The method of claim 21, wherein the statin is simvastatin or lovastatin.

23. The method of claim 20, wherein the statin reduces an inflammatory response at the site of the vulnerable plaque.

24. The method of claim 23, wherein the statin is atorvastatin, pitavastatin or fluvastatin.

25. The method of claim 20, wherein the statin reduces proliferation or migration of smooth muscle cells.

26. The method of claim 25, wherein the statin is lovastatin, simvastatin, atorvastatin, or fluvastatin.

27. The method of claim 20, wherein the statin is a hydrophobic statin.

28. The method of claim 27, wherein the statin is lovastatin, simvastatin, atorvastatin, or fluvastatin.

29. The method of claim 15, wherein the first polymer coating is absorbable and degrades at a pre-determined rate.

30. The method of claim 29, wherein the device further comprises a top coating comprising and absorbable polymer material and an anti-thrombogenic agent.

31. A method of treating vulnerable plaque comprising:
inserting a device into the individual,
wherein the device comprises a therapeutically effective amount of a drug selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an anti-thrombogenic agent, a drug that promotes healing and re-endothelialization, and combinations thereof.

32. A device or treating vulnerable plaque comprising:
a generally tubular body comprising a contracted state and an enlarged state, an interconnecting structure with pores substantially along the length of the generally tubular body, wherein the generally tubular body is expandable from the contracted state to the enlarged state, and wherein the generally tubular body is sufficiently flexible such that the tubular body conforms to a contour of an inner surface of a body lumen, and
a therapeutically effective amount of a drug selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an ant-thrombogenic agent, a drug that promotes healing and re-endothelialization, and combinations thereof.

33. The device of claim 32, wherein the generally tubular body is self-expandable.

34. The device of claim 32, wherein the generally tubular body further comprises a plurality of filaments coherently engaged by braiding, weaving, or knitting.

35. The device of claim 32, wherein the pore size is no greater than about 500 microns.

36. The device of claim 32, wherein the interconnecting structure further comprises a plurality of polymer or metallic microfilaments.

37. The device of claim 36, wherein the drug is contained within a plurality of polymer microfilaments.

38. The device of claim 36, wherein the drug is contained within a plurality of metallic microfilaments.

39. The device of claim 32, wherein the drug is contained in a polymer coating.

40. The device of claim 39, wherein the polymer coating reduces thrombosis.

41. The device of claim 39, wherein the polymer coating coats the interconnecting structure.

42. The device of claim 39, wherein the polymer coating is coated with a second polymer coating that increases or decreases the release rate of the drug.

43. The device of claim 39, wherein the polymer coating is absorbable and degrades at a pre-determined rate.

44. The device of claim 32, further comprising a top coating comprising an absorbable polymer material and an anti-thrombogenic agent.

45. The device of claim 32, further comprising a therapeutically effective amount of a statin.

46. The device of claim 45, wherein the statin promotes endothelial cell function.

47. The device of claim 46, wherein the statin is simvastatin or lovastatin.

48. The device of claim 45, wherein the statin reduces an inflammatory response at a site of a vulnerable plaque.

49. The device of claim 48, wherein the statin is atorvastatin or fluvastatin.

50. The device of claim 45, wherein the statin reduces proliferation or migration of smooth muscle cells.

51. The device of claim 50, wherein the statin is lovastatin, simvastatin, atorvastatin, or fluvastatin.

52. The device of claim 45, wherein the statin is a hydrophobic statin.

53. The device of claim 52, wherein the statin is lovastatin, simvastatin, atorvastatin, or fluvastatin.

54. A device for treating vulnerable plaque comprising:
i) a body lumen support structure,
ii)a first polymer coating disposed on the body lumen support structure, and
iii) a therapeutically effective amount of a drug disposed within the first polymer coating, where the drug is selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an anti-thrombogenic agent, a drug that promotes healing and re-endothelialization, and combinations thereof.

55. The device of claim 54, wherein the body lumen support structure comprises a plurality of fibers having an average diameter less than about 100 microns, wherein the fibers are arranged in a substantially random pattern on the body lumen support structure for creating a plurality of substantially random interstitial spaces within the body lumen support structure.

56. The device of claim 55, wherein the plurality of fibers are arranged in a substantially random pattern by a process comprising electrospinning.

57. The device of claim 54, wherein the first polymer coating is coated with a second polymer coating that increases or decreases the release rate of the drug from the first polymer coating.

58. The device of claim 54, wherein the device is a stent.

59. The device of claim 54, further comprising a therapeutically effective amount of a statin.

60. The device of claim 59, wherein the statin promotes endothelial cell function.

61. The device of claim 60, wherein the statin is simvastatin or lovastatin.

62. The device of claim 59, wherein the statin reduces an inflammatory response at a site of a vulnerable plaque.

63. The device of claim 62, wherein the statin is atorvastatin or fluvastatin.

64. The device of claim 59, wherein the statin reduces proliferation or migration of smooth muscle cells.

65. The device of claim 64, wherein the statin is lovastatin, simvastatin, atorvastatin, or fluvastatin.

66. The device of claim 59, wherein the statin is a hydrophobic statin.

67. The device of claim 66, wherein the statin is lovastatin, simvastatin, atorvastatin, or fluvastatin.

68. The device of claim 54, wherein the first polymer coating is absorbable and degrades at a pre-determined rate.

69. The device of claim 54, further comprising a top coating comprising absorbable polymer material and an anti-thrombogenic agent.

70. A device for treating vulnerable plaque comprising a therapeutically effective amount of a drug selected from the group consisting of a statin, an angiotensin converting enzyme (ACE) inhibitor, a metalloproteinase inhibitor, 17-β-estradiol, heparin, chemically-modified heparin, a non-statin lipid-lowering drug, an antioxidant, a β-adrenergic blocker, an anti-inflammatory immunomodulator, an anti-proliferative drug, a drug that inhibits cellular migration, a drug that promotes extracellular matrix (ECM) synthesis or inhibits ECM degradation, a drug that reduces hyperplasia, an anti-thrombogenic agent, a drug that promotes healing and re-endothelialization, and combinations thereof, wherein the device is capable of being inserted into a vessel of an animal.
